(19) United States
(12) Patent Application Publication
(10) Pub. No.: US 2023/0172544 A1
(43) Pub. Date: Jun. 8, 2023

(54) WEARABLE PHYSICAL HEALTH TESTING SYSTEMS AND ASSOCIATED DEVICES AND METHODS

(71) Applicant: Reperio Health, Inc., Portland, OR (US)

(72) Inventors: Travis Benjamin Rush, Happy Valley, OR (US); Matthew Robert Wallington, Oregon City, OR (US)

(21) Appl. No.: 17/922,343

(22) PCT Filed: Apr. 30, 2021
(86) PCT No.: PCT/US2021/030330

§ 371 Date: Oct. 28, 2022

Related U.S. Application Data
(60) Provisional application No. 63/019,154, filed on May 1, 2020.

(51) Int. Cl. A61B 5/00; G16H 40/67; G16H 15/00; A61B 5/256; A61B 5/282; A61B 5/0205; A61B 5/276

(52) U.S. Cl. CPC A61B 5/6804; G16H 40/67; G16H 15/00; A61B 5/256; A61B 5/282; A61B 5/6843; A61B 5/0205; A61B 5/276; A61B 2562/0217; A61B 7/04

(57) ABSTRACT

Wearable physical health testing systems and associated devices and methods are disclosed herein. A wearable system configured in accordance with embodiments of the present technology can include, for example, a communications hub, and a plurality of physical health testing devices. The communications hub and the plurality of physical health testing devices can integrated into an article of clothing, such as a jacket, a shirt, or a body suit. The physical health testing devices are in wired and/or wireless communication with the communications hub. Each physical health testing device is configured to generate physical health data of a user and to transmit generated physical health data to the communication hub and/or a user's mobile device. The wearable system provides an automated physical exam that can be performed at user's homes or other convenient locations.

### CROSS-REFERENCE TO RELATED APPLICATION(S)

This application claims the benefit of U.S. Provisional Patent Application No. 63/019,154, filed May 1, 2020, which is incorporated by reference herein in its entirety.

### TECHNICAL FIELD

The present disclosure relates generally to systems for providing physical health screening exams. More specifically, the present disclosure relates to wearable physical health testing systems and associated devices and methods.

### BACKGROUND

A physical exam, or “physical,” is a screening test that is executed by a healthcare professional, such as a primary care provider, in a medical office to assess a person's overall health. A physical exam evaluates patient vitals and other diagnostic parameters to detect various medical conditions, identify potential medical issues that may be of concern in the future, track changes in a person's physical health over time, and/or determine whether an individual requires further medical tests. As such, routine physical exams (e.g., once or twice per year) are essential for tracking an individual's medical history and detecting potential health concerns in early stages.

Despite the clear benefits, many individuals do not undergo routine physical exams for a variety of reasons. These include, among other reasons, travel time to physician's office, inconvenience of scheduling and going to a doctor's appointment, difficulty getting time off work, financial uncertainty of the cost of a hospital visit, and/or low urgency or priority, especially when the individual currently feels fine. Furthermore, physicals are not always prioritized by healthcare providers and systems due to the inherent lack of acuity, the overall needs of medically underserved areas, and/or low reimbursement rates. Delaying or eliminating routine physical exams, however, decreases the likelihood of early diagnosis and intervention, and decreases patient medical history that can be important for diagnosis and treatment when it becomes necessary.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. The drawings should not be taken to limit the disclosure to the specific embodiments depicted, but are for explanation and understanding only.

FIG. 1 is a system diagram of an environment for operating a wearable physical health testing system in accordance with various embodiments of the present technology.

FIG. 2A is a front view of a wearable physical health testing system configured in accordance with various embodiments of the present technology.

FIG. 2B is a back view of the wearable physical health testing system of FIG. 2A configured in accordance with various embodiments of the present technology.

FIG. 3 is a flow diagram illustrating a physical exam routine using a wearable physical health testing system configured in accordance with various embodiments of the present technology.

### DETAILED DESCRIPTION

The following disclosure describes wearable physical health testing systems and associated devices and methods. In particular, the following disclosure describes a wearable physical health testing system that includes a plurality of physical health testing devices for a user to perform a physical exam on his/her own and/or apart from a healthcare professional. As an example, the wearable physical health testing systems disclosed herein can include an article of clothing, such as a jacket, a shirt, or a body suit, that integrates one or more adjustment cables, one or more adjustment motors, a plurality of physical health testing devices, and a communications module for communicating with external devices. This integrated, wearable system can interface with a software application (e.g., a mobile application) on a user device and/or one or more remote servers/databases. In some embodiments, the physical health testing devices include one or more blood pressure and/or heart rate cuffs and/or monitors, a plurality of ECG electrodes, one or more stethoscope microphones, and/or other physical health testing devices. Using the physical health testing devices, the system performs an automated protocol of physical health tests to generate data related to a user's health that can be assessed and used by medical professionals and form a part of the patient's medical history.

The physical health testing devices are in wired and/or wireless communication with the communications hub. In turn, the hub is in wired or wireless communication with the software application running on a user's electronic device, which can provide instructions and interfaces that guide the user through a series of tests (e.g., automated tests) provided by the integrated, wearable physical exam system. As the plurality of physical health testing devices generate health data related to the user, the testing devices communicate the health data to the hub, and the hub associates the health data with the user's account and/or stores the health data for future reference/review. Additionally, or alternatively, the testing devices communicate health data to the user via the software application and/or via indicators on the wearable system. In some embodiments, the physical health testing system can provide information to the user (e.g., via the mobile application) related to the recorded data, such as whether certain measurements are in a predetermined “healthy” or “normal” range and/or provide recommendations related to the recorded data that the user consult a healthcare professional (e.g., when the detected health data is outside of the predetermined normal range). In these and other embodiments, a user can send the health data to a healthcare professional via the physical health testing system and/or a user device running the software application, and/or can generate a code for the user to share with a healthcare professional who can use the code to retrieve health data generated during the physical exam(s) and corresponding to the user.

Because the wearable physical health testing system is portable, a user can run a variety of physical health tests at a convenient time and from a convenient location, such as at home, work, a hotel, an assisted living facility, gym, and/or school. This reduces or obviates the practice and inconvenience of scheduling and attending a doctor's appointment for a physical exam. Furthermore, because the physical exams can be performed outside of a medical facility and/or without supervision of a healthcare professional, the costs of the physical exams are predictable rather than the variable post-appointment invoices associated with doctor's office visits. In addition, the physical health testing systems are expected to free up healthcare time, funds, and resources for other needs (e.g., acute or complex patient care, research, etc.). In turn, users are more likely to stay current on their routine physical exams, increasing the chances of early detection of medical concerns and generating a wealth of medical history data for healthcare professionals to consult when a patient does become ill.

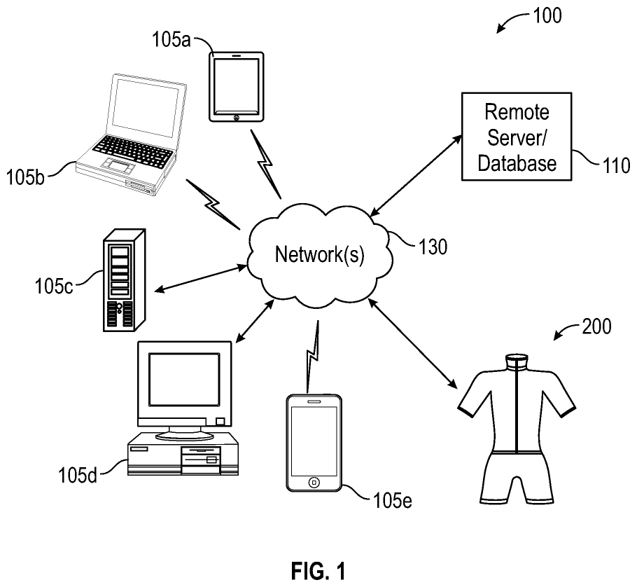

Certain details are set forth in the following description and in FIGS. 1-3 to provide a thorough understanding of various embodiments of the disclosure. However, other details describing well-known structures and systems often associated with physical health testing systems and associated methods are not set forth below to avoid unnecessarily obscuring the description of various embodiments of the disclosure.

Many of the details, dimensions, angles, and other features shown in FIGS. 1-3 are merely illustrative of particular embodiments of the disclosure. Accordingly, other embodiments can have other details, dimensions, angles, and features without departing from the spirit or scope of the present disclosure. In addition, those of ordinary skill in the art will appreciate that further embodiments of the disclosure can be practiced without several of the details described below.

### A. SELECTED EMBODIMENTS OF WEARABLE PHYSICAL HEALTH TESTING SYSTEMS AND ASSOCIATED DEVICES AND METHODS

FIG. 1 is a system diagram of an environment 100 in which a wearable physical health testing system 200 (“the wearable system 200” or “the system 200”) operates, configured in accordance with various embodiments of the present technology. In the environment 100, the system 200 can connect to (e.g., wirelessly and/or via one or more wires) and/or communicate with one or more devices 105 (identified individually as 105a-e in FIG. 1) over one or more networks 130, including public or private networks (e.g., the internet). The one or more devices 105 can include personal computers, server computers, handheld or laptop devices, cellular telephones, wearable electronics, tablet devices, multiprocessor systems, microprocessor-based systems, set-top boxes, programmable consumer electronics, network PCs, minicomputers, mainframe computers, distributed computing environments that include any of the above systems or devices, or the like. In these and other embodiments, the one or more devices 105 can include other remote or local devices, such as landline phones, fax machines, medical devices, thermostats, speakers, and other devices.

As shown in FIG. 1, the wearable system 200 can connect to and/or communicate with one or more remote servers/databases 110. In some embodiments, a remote server/database 110 can be an edge server which receives client requests and coordinates fulfillment of those requests through other servers. The remote servers/databases 110 can comprise computing systems. Although the remote servers/databases 110 are displayed logically as a single server/database, the remote servers/databases 110 can be a distributed computing environment encompassing multiple computing devices and/or databases located at the same or at geographically disparate physical locations. In some embodiments, the remote servers/databases 110 correspond to a group of servers.

In some embodiments, the one or more devices 105, the system 200, and/or the remote servers/databases 110 can each act as a server or client to other server/client devices. The remote servers/databases 110 can include one or more databases. The one or more databases can warehouse (e.g. store) information such as health educational lessons, health information, various alerts or warnings, user accounts/profiles, generated health data, drivers/software necessary to operate certain applications and/or devices, and/or other information.

The one or more networks 130 allow for communication in the environment 100. The one or more networks 130 can include one or more wireless networks, such as, but not limited to, one or more of a Local Area Network (LAN), Wireless Local Area Network (WLAN), a Personal Area Network (PAN), Campus Area Network (CAN), a Metropolitan Area Network (MAN), a Wide Area Network (WAN), a Wireless Wide Area Network (WWAN), Global System for Mobile Communications (GSM), Personal Communications Service (PCS), Digital Advanced Mobile Phone Service (D-Amps), Bluetooth, Wi-Fi, Fixed Wireless Data, 2G, 2.5G, 3G, 3.75G, 4G, 5G, LTE networks, enhanced data rates for GSM evolution (EDGE), General packet radio service (GPRS), enhanced GPRS, messaging protocols such as, TCP/IP, SMS, MMS, extensible messaging and presence protocol (XMPP), real time messaging protocol (RTMP), instant messaging and presence protocol (IMPP), instant messaging, US SD, IRC, or any other wireless data networks or messaging protocols. Network 130 may also include wired networks.

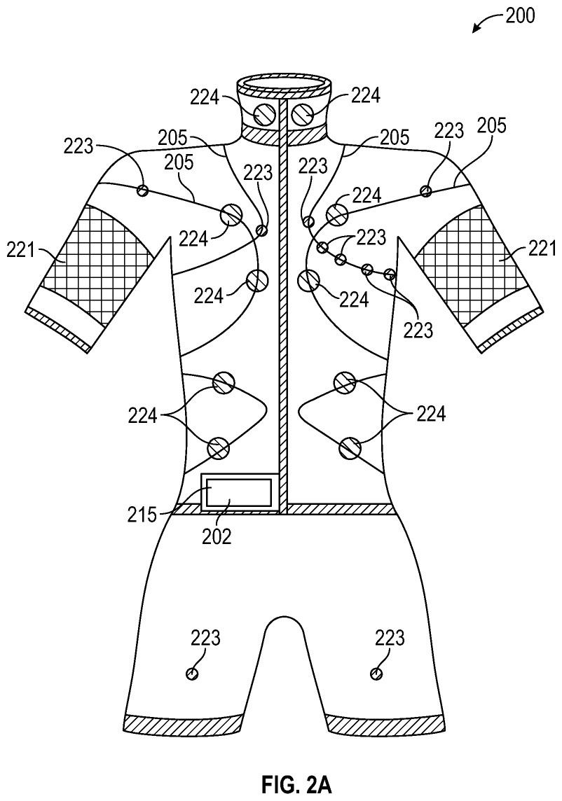

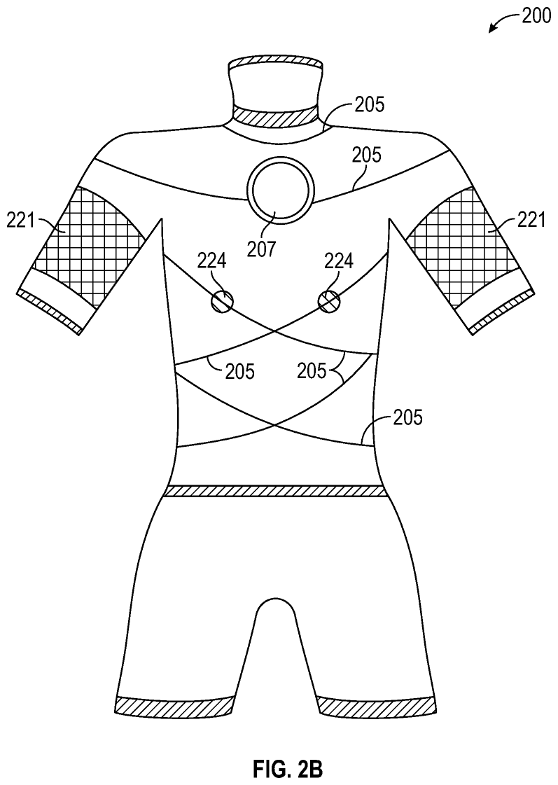

FIG. 2A is a front view and FIG. 2B is a back view of a wearable physical health testing system 200 configured in accordance with various embodiments of the present technology. As shown in FIGS. 2A and 2B, the wearable system 200 is an article of clothing (in this case, a jacket, a shirt, or a body suit) that integrates a power supply 202, a communications hub 215, and a plurality of physical health testing devices (e.g., blood pressure cuff(s) 221, electrocardiogram (ECG) electrodes 223, stethoscope microphones 224, and/or other physical health testing devices). As described in greater detail below, the wearable system 200 can further include adjustment cable(s) 205 and/or corresponding adjustment motor(s) 207 that ensure the wearable system 200 is snugly fit around a user (e.g., to ensure proper positioning of the physical health testing devices and/or to ensure the physical health testing devices obtain accurate health measurements of the user).

In some embodiments, a portion of the system 200 is constructed from an elastic material, such as spandex, stretch vinyl, nylon, and/or other (e.g., blends of) materials. In this regard, the system 200 can be form fitting at least around a user's legs. In these and other embodiments, portions of the system 200 can be made of other (e.g., elastic, semi-elastic, or non-elastic material(s)), such as one or more sport garment materials including synthetic fabric, miracle microfiber, cotton, calico, and/or other materials.

The system 200 includes a plurality of physical health testing devices and/or related objects. As shown, the plurality of physical health testing devices includes blood pressure/heart rate cuff(s) and/or monitor(s) 221, a plurality of electrocardiogram (ECG) electrodes 223 (FIG. 2A), and one or more stethoscope microphones 224. In some embodiments, the system 200 can include additional physical health testing devices and/or related objects (not shown) in addition to or in lieu of the physical health testing devices and related objects illustrated in FIGS. 2A and 2B. For example, the system 200 can include one or more temperature sensors and/or one or more electrically conductive gel packets. The gel packet(s) can be configured to fit within slots or holding pouches/pockets beneath or adjacent one or more of the ECG electrodes 223 on the inside of the system 200. As the system 200 compresses against a user's body (e.g., using the adjustment motor(s) 207 and cable(s) 205, as described in greater detail below), the gel packets can break and distribute electrically conductive gel onto the user's skin and/or beneath one or more ECG electrodes 223 to facilitate creating an electrical connection between the user's body and a corresponding ECG electrode 223. In these and other embodiments, the additional physical health testing devices and/or related objects can include a scale, a thermometer, vision testing systems, dermatological screening systems, cameras, blood testing systems, Band-aids, and/or other first aid medical supplies (e.g., gauze).

In these and still other embodiments, the additional physical health testing devices and/or related objects can include one or more “on”/“start” button(s), one or more “off”/“stop” button(s), one or more “pause”/“resume” button(s), and/or one or more display(s) (e.g., for providing a user visual feedback, such as indications of health data related to the user, color-coded feedback that measured health data falls within or falls outside of a corresponding normal and healthy range, and/or indications of successful pairing with the communications hub 215 and/or a user device 105 (FIG. 1)). The “on”/“start” button(s), the “off”/“stop” button(s), the “pause”/“resume” button(s), and/or the display(s) may be integrated into corresponding physical health testing devices, the system 200, and/or one or more displays viewable on the user device 105. In some embodiments, all or a subset of the physical health testing devices and/or related objects of the plurality of physical health testing devices and/or related objects included in the wearable system 200 are approved for use by an appropriate governmental administrative body (e.g., in the United States—by the U.S. Food and Drug Administration of the United States Department of Health and Human Services).

The hub 215 of the system 200 can include a receiver (not shown), a transmitter (not shown), and a controller/processor (not shown), among other circuitry components. In operation, the hub 215 is configured to provide communication between individual physical health testing devices, a software application running on a user's device 105 (FIG. 1), and/or one or more remote servers/databases 110 (FIG. 1) or other devices 105. In this regard, individual ones of the plurality of physical health testing devices, the device(s) 105, and/or the remote servers/databases 110 can communicate with the hub 215 over a wired or wireless connection. For example, individual ones of the plurality of physical health testing devices, the device(s) 105, and/or the remote servers/databases 110 can be paired with the hub 215 and can communicate with the hub 215 over a Wi-Fi, Bluetooth, Bluetooth Low Energy (“BLE”), Zigbee, hardwire, and/or other communication means. As a more specific example, individual ones of the plurality of physical health testing devices can communicate device initialization/startup information, status data, health data related to a user, and/or other information directly to the hub 215 (e.g., via a Bluetooth connection). In turn, the hub 215 can communicate all or a subset of the information (e.g., health data) to one or more devices 105 (e.g., a user's mobile device 105e that is currently running a related software application) paired with the hub 215 and/or to one or more remote servers/databases 110 (e.g., for storage in database entries associated with a user). Additionally, or alternatively, individual ones of the plurality of physical health testing devices can communicate generated health data directly to one or more connected devices 105, which in turn can communicate all or a subset of the received health data to the hub 215 and/or to one or more remote servers/databases 110.

More specifically, the hub 215 is configured to communicate with one or more peripheral devices, circuits, and/or components of the system 200. For example, the hub 215 can be configured to execute instructions stored in memory, including various processes, logic flows, and routines for controlling operation of the system 200 and/or for managing communications between the various electrical circuits and devices on and/or connected to the system 200. In some embodiments, memory used to store the instructions can include electrically erasable programmable read-only memory (“EEPROM”), double data rate (any generation) dynamic random-access memory (“DDR DRAM”), and/or NAND flash memory (“NAND flash”). The EEPROM, for example, can be configured to store boot instructions of the system 200. The DDR DRAM can permit high speed data transfers while the system 200 remains powered on and/or while power is supplied to the system 200. The NAND flash can provide non-volatile memory storage (e.g., to store system, user, and/or other information).

Peripheral devices, circuits, and/or components in communication with the hub 215 can include various communication devices, circuits, and/or components on and/or connected to the system 200. For example, the system 200 can include Wi-Fi and/or Bluetooth controller(s). A Wi-Fi controller (e.g., an IEEE 802.11 b/g/n/RF/Baseband/Medium Access Control (MAC) link controller) can allow the system 200 to wirelessly connect to the internet. In some embodiments, the Wi-Fi controller can wirelessly connect to the internet by leveraging TV white space channels. A Bluetooth controller (e.g., a Bluetooth 4.0 compliant module or controller) can allow the system 200 to communicate with Bluetooth compatible devices. In some embodiments, the Bluetooth module can be optimized for low power consumption. In some embodiments, the system 200 can include an Wi-Fi and/or Bluetooth antenna to improve signal strength.

The system 200 can include one or more adjustment mechanisms that cinch the wearable system or portions thereof such that it fits snugly against the user's body. In the embodiment illustrated in FIGS. 2A and 2B, for example, the system 200 includes one or more cinch or adjustment cables 205 and one or more cinch or adjustment motors 207 (FIG. 2B). The adjustment motor(s) 207 of the system 200 are configured to draw in the adjustment cable(s) 205 to compress the system 200 until the system 200 is held tight against a user's body, thereby adjusting a default shape and size of the system 200 to a shape and size matching the user's body. For example, the adjustment motor(s) 207 can draw in the adjustment cable(s) 205 until the adjustment motor(s) 207 detect one or more threshold resistances, indicating that a corresponding portion of the system 200 is tight against a corresponding portion of the user's body. This can facilitate proper alignment and contact between one or more physical health testing devices (e.g., one or more of the ECG electrodes 223 and/or one or more of the stethoscope microphones 224) and corresponding portions of the user's body. In some embodiments, the adjustment motor(s) 207 can track an amount of cable drawn in a given direction. In turn, the adjustment motor(s) 207, the hub 215, a user device 105 (FIG. 1), and/or remote servers/databases 110 (FIG. 1) can determine a shape and size of a corresponding portion of the user's body (e.g., of the user's torso). In some embodiments, the adjustment motor(s) 207 and/or the adjustment cable(s) 205 can be operably coupled to one or more displays viewable on the system 200 and/or on a user device 105.

The system 200 can further include a power supply 202, such as one or more (e.g., rechargeable) batteries and/or associated charge ports/wires. The power supply 202 can be configured to deliver power to the hub 215, the individual ones of the plurality of physical health testing devices, and/or the activation mechanism (e.g., the adjustment motors 207) for cinching the wearable system around the user. In some embodiments, one or more of the hub 215, the activation mechanism, and/or the health testing devices may include integrated power supplies (not shown) that supply energy to the associated device or subset of the devices. Additionally, or alternatively, the system 200 can include charge ports/wires configured to charge one or more batteries of one or more of the physical health testing devices separately from the power supply 202 of the hub 215.

The heart rate and/or blood pressure cuff(s) and/or monitor(s) 221 (“the BP/HR cuff 221”) of the system 200 are configured to measure a user's heart rate, systolic blood pressure, and/or diastolic blood pressure. In particular, each BP/HR cuff 221 includes an arm strap at least partially integrated into a sleeve of the system 200 and/or otherwise attached thereto. When the system 200 is worn by a user, the BP/HR cuff 221 compresses about the user's arm and conducts several readings to determine the user's heart rate and/or blood pressure. The BP/HR cuff 221 then communicates all or a subset of this health data to the hub 215 and/or to the user's device 105 (e.g., directly and/or via the hub 215) running a related software application. In some embodiments, a BP/HR cuff 221 can include a display (not shown) configured to provide visual feedback (not shown) of a user's blood pressure and/or heart rate during use of the BP/HR cuff 221. In these and other embodiments, the display can provide other visual feedback, such as an indication (not shown) that the BP/HR cuff 221 has been successfully paired with the hub 215 (FIG. 2A) and/or with a user's device 105 (FIG. 1) running the related software application. In some embodiments, the display may be integrated into one or more displays viewable on the wearable system and/or provided on the user device 105.

The plurality of ECG electrodes 223 of the system 200 are configured to measure electrical activity of a user's heart. As discussed above, the adjustment motor(s) 207 and the adjustment cable(s) 205 are configured to compress the wearable system to position the plurality of ECG electrodes 223 at a proper position on the user's body to ensure the ECG electrodes 223 capture an accurate ECG reading of the user's heart. In some embodiments, the system 200 can include more ECG electrodes 223 than necessary to capture an ECG of the user's heart. For example, the system 200 can include one or more redundant ECG electrodes 223 at one or more locations on the system 200. In these embodiments, each of the ECG electrodes 223 (including the redundant ECG electrodes 223) at a corresponding location on the system 200 can be configured to capture an ECG reading of the user's heart. In turn, the corresponding ECG electrodes 223, the hub 215, a user device 105 (FIG. 1), and/or the remote servers/databases 110 (FIG. 1) can determine which of the ECG electrodes 223 are positioned against an appropriate location of the user's body and/or are capturing accurate ECG data of the user's heart. This can facilitate positioning ECG electrodes 223 at the same or similar locations on two different users' bodies regardless of differences between the shapes and sizes of the users' bodies. Additionally, or alternatively, this can ensure a proper and/or accurate ECG reading in the event that one of the ECG electrodes 223 is not operating correctly (e.g., has malfunctioned or is unable to create an electrical connection with a user's body, for example, because a corresponding gel packet did not break or distribute electrically conductive gel properly). After the ECG electrodes 223 record ECG data of a user's heart, the ECG electrodes 223 communicate all or a subset of this health data to the hub 215 and/or to the user's device 105 (e.g., directly and/or via the hub 215) running a related software application for further processing and analysis. In some embodiments, the ECG electrodes 223 can be operably coupled to one or more displays viewable on the system 200 and/or on a user device 105.

The one or more stethoscope microphones 224 of the system 200 are configured to record sounds and murmurs made by a user's heart and lungs to generate a phonocardiogram (PCG). As discussed above, the adjustment motor(s) 207 and the adjustment cable(s) 205 are configured to compress the wearable system to position the stethoscope microphones 224 at appropriate positions on the user's body to ensure the stethoscope microphones 224 capture an accurate PCG recording of the user's heart and lungs. In some embodiments, the system 200 can include more stethoscope microphones 224 than necessary to capture a PCG of the user's heart and lungs. For example, the system 200 can include one or more redundant stethoscope microphones 224 at one or more locations on the system 200. In these embodiments, each of the stethoscope microphones 224 (including the redundant stethoscope microphones 224) at a corresponding location on the system 200 can be configured to capture a PCG recording of the user's heart and/or lungs. In turn, the corresponding stethoscope microphones 224, the hub 215, a user device 105 (FIG. 1), and/or the remote servers/databases 110 (FIG. 1) can determine which of the stethoscope microphones 224 are positioned against an appropriate location of the user's body and/or are capturing accurate PCG data of the user's heart and lungs. This can facilitate positioning stethoscope microphones 224 at the same or similar locations on two different users' bodies regardless of differences between the shapes and sizes of the users' bodies. Additionally, or alternatively, this can ensure a proper and/or accurate PCG recording in the event that one of the stethoscope microphones 224 is not operating correctly (e.g., has malfunctioned and/or is not receiving relevant PCG data). After the stethoscope microphones 224 record PCG data of a user's heart and lungs, the stethoscope microphones 224 communicate all or a subset of this health data to the hub 215 and/or to the user's device 105 (e.g., directly and/or via the hub 215) running a related software application for further processing and analysis. In some embodiments, the stethoscope microphones 224 can be operably coupled to one or more displays viewable on the system 200 and/or on a user device 105.

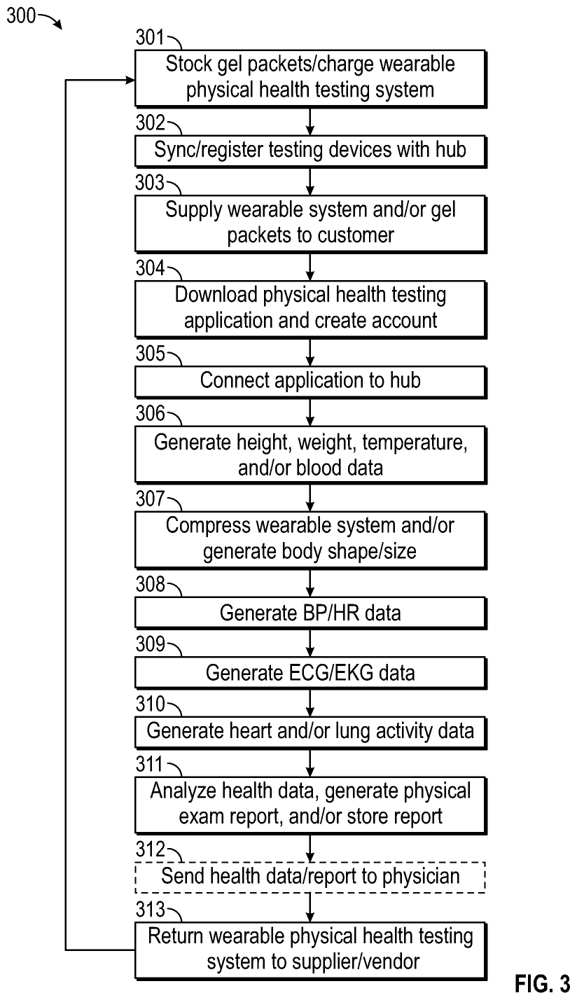

FIG. 3 is a flow diagram illustrating an automated physical exam routine 300 using a physical health testing system configured in accordance with various embodiments of the present technology. In some embodiments and as described below, the routine 300 can be executed, at least in part, by various components of the system 200 described above with reference to FIGS. 1-2B. In these and other embodiments, the routine 300 can be executed, at least in part, by one or more devices 105 (FIG. 1), such as by a user's mobile device 105e running a related software application. In these and still other embodiments, the routine 300 can be executed, at least in part, by one or more remote servers/databases 110 (FIG. 1), by a user of the system 200, and/or by a supplier or vendor of the system 200.

For the sake of example only, the routine 300 is discussed in detail below in the context of one or more users who subscribe to a service offered by a supplier or vendor. The service in this example is an agreement to supply (e.g., send in the mail) a wearable physical health testing system to a user (e.g., one, twice, or more per year). After creating an account associated with the service, the user performs a physical exam each time he/she is supplied the wearable system to generate data corresponding to his/her physical health. Once the user has completed the physical exam, the user returns the wearable system (e.g., in the mail) to the supplier or vendor. In this manner, users are able to run a variety of physical health tests from any location (e.g., at home, at work, at a hotel, at an assisted living facility, at a gym, at school) and/or at a time that is convenient for him/her, thereby obviating the practice and inconvenience of scheduling and attending a doctor's appointment for a physical exam. Furthermore, because the physical exam can be performed outside of a hospital or medical facility and/or without supervision of a healthcare professional, users can undergo a physical exam without unexpected hospital bills. In addition, the physical health testing systems frees up healthcare time, funds, and resources to be spent on treating patients with more acute needs. In turn, users are more likely to stay current on their routine physical exams, increasing the chances of early detection of medical concerns and generating a wealth of medical history data for healthcare professionals to consult when a patient does become ill. To incentive participation in the service, insurance companies can offer a discount on participating users' or companies' health insurance premiums (e.g., equal to or greater than the cost of subscribing to the service).

In other embodiments, users can purchase a wearable system outright. In these embodiments, the service can be an agreement to supply (e.g., send in the mail, supply in stores, etc.) one or more gel packets or other related objects (e.g., a software subscription) to a user. After creating an account associated with the service, the user can perform a physical exam after loading the gel packets into the wearable system to generate data corresponding to his/her physical health. Rather than returning the wearable system to the supplier or vendor, the user can wash the wearable system and/or can reload the wearable system with gel packets before conducting another physical exam using the same wearable system.

At block 301, the routine 300 begins by stocking and/or charging a wearable physical health testing system (e.g., one or more physical health testing devices, a communications hub, and/or related objects). In some embodiments, the routine 300 can stock a wearable physical health testing system by loading one or more gel packets into slots and/or pouches/pockets built on the inside of the wearable system and/or into packaging used to ship the wearable system to subscribing users. In these and other embodiments, the routine 300 can stock a wearable physical health testing system by cleaning and sterilizing the wearable system. In these and still other embodiments, the routine 300 can stock the wearable system by cleaning and sterilizing related objects included with the wearable system, such as thermometers, blood testing devices, scales, etc., and/or by removing waste (e.g., hazardous waste generated from blood tests) included in a disposal container (not shown) accompanying the wearable system.

The routine 300 can charge the wearable physical health testing system by replacing batteries in the wearable system and/or by charging rechargeable batteries included within the wearable system. In turn, the batteries of the system can charge the batteries of individual physical health testing devices included in the system. In these and other embodiments, the routine 300 can charge the wearable system by replacing batteries and/or by directly charging one or more rechargeable batteries of the individual physical health testing devices. Alternatively, one or more components (e.g., the communications hub, individual physical health testing devices, etc.) can be powered by connecting the wearable system to a power supply (e.g., by plugging a corresponding power cord into a power outlet).

At block 302, the routine 300 continues by syncing or registering the communications hub of the wearable system with the physical health testing devices and/or with a user's account. In some embodiments, the routine 300 syncs the physical health testing devices with the hub by connecting the physical health testing devices to the hub over a network (e.g., using one or more wired connections and/or Wi-Fi, Bluetooth, or another wireless communication protocol) such that the physical health testing devices are placed in wired and/or wireless in communication with the hub and are thereby registered to the hub. In these and other embodiments, the routine 300 registers the wearable system (e.g., the hub of the wearable system) to a specific user's account (e.g., such that the wearable system is operable only by the user and only when the user (i) logs into his/her account on a related software application and (ii) connects the software application to the communications hub of the wearable system).

At block 303, the routine 300 continues by supplying the wearable physical health testing system to a user. In some embodiments, the routine 300 can supply the wearable system to the user by shipping the wearable system to the user (e.g., at the user's home or at another location). In these and other embodiments, the routine 300 can supply the wearable system to the user via other means (e.g., by making the wearable system available for checkout to the user, such as at a pharmacy or hospital).

At block 304, the routine 300 when the user downloads a software application related to the wearable physical health testing system onto a user's device. For example, the user can download a mobile application or another software application to a user's mobile device (e.g., at the direction of the user). In some embodiments, the wearable system includes directions or a QR code to aid the user in downloading the software application. Once downloaded, the software application can require the user to create and/or log into their account. The account can be unique to the user such that any health data generated by the wearable system can be associated with the user via the account and stored for future reference, analysis, and/or review.

At block 305, the routine 300 continues by connecting the software application to the hub of the wearable system. In some embodiments, the routine 300 connects the software application to the hub over a network (e.g., using one or more wired connections and/or Wi-Fi, Bluetooth, or another wireless communication protocol) such that the software application on the user's device is placed in communication with the hub. As a specific example, the software application running on the user's device can instruct the device to scan for and connect to the wearable system via Bluetooth. In response, the wearable system can provide the device a notification that connection was successful. In some embodiments, the user device can request (and the wearable system can provide) connection characteristic information (e.g., Bluetooth Low Energy characteristic information). After the user device connects to the wearable system, the software application can subscribe to status and test result notifications of specific physical health testing devices included in the wearable system (e.g., all of the physical health testing devices included in the wearable system or only those physical health testing devices that correspond to the user's account). Example notifications include battery status info of the hub and/or of one or more of the physical health testing devices, firmware information of the physical health testing devices and/or of sensors included in the physical health testing devices, and/or data (e.g., health results and/or errors) generated during one or more physical health tests of the physical exam.

Because the physical health testing devices of the wearable system are registered with and are in communication with the hub (block 302), the routine 300 also places the software application on the user's device in communication with the physical health testing devices (via notifications sent to the software application from the hub) when the software application is connected to the hub. The software application can remain connected with the communications hub for the duration of the physical exam. In other embodiments, the software application can intermittently connect to the hub (e.g., when the user initiates one of the physical health tests of the physical exam, after a user indicates that one or more physical health tests of the physical exam are complete, at the end of the physical exam, etc.). In these and other embodiments, the routine 300 can connect the software application directly to individual physical health testing devices of the wearable system, and/or the routine 300 can connect the software application to one or more remote servers/databases (e.g., directly or via the hub). By connecting the physical health testing devices to the hub of the wearable system, the routine 300 obviates the practice of a user downloading a software application on his/her device (e.g., device 105 illustrated in FIG. 1) dedicated to each individual physical health testing device included in the wearable system.

As discussed in greater detail below, the software application can provide a user step-by-step instructions for performing a physical exam using the physical health testing devices and related objects included in the wearable system. In particular, the software application can permit a user to control individual physical health testing devices of the wearable system, view reports of health data related to the user and generated using the physical health testing devices, store all or a subset of generated health data (e.g., to one or more remote servers/databases and/or associated with the user's account), view healthcare recommendations, and/or share generated health data with a healthcare professional. More specifically, the wearable system, in operation, is configured to provide a variety of services to a user and/or to facilitate use of one or more functions, some of which are discussed in greater detail below.

In some embodiments, the user can view, access, and/or interface with several of these services and/or functions via a user interface (UI) of the software application that can be presented on a screen of the user's device when the software application is open and/or connected to the hub. The UI can include one or more buttons or menu options that correspond to one or more of the physical health tests, services, and/or functions of the system. For example, the UI can include instructions for performing physical health tests; options to start, stop, or skip tests; indicators for whether health data is being generated, for how long to continue generating/collecting health data for a given physical health test, and/or for when health data has been successfully received by the hub or by the software application on the user's device; and/or options to view, share, and/or save generated health data reports. The software application (including a variety of user interfaces that can be displayed to the user via the software application), communications between the software application and the hub, and communications between the hub and various physical health testing devices of the wearable system are discussed in greater detail in U.S. patent application Ser. No. 17/085,293, which is incorporated by reference herein in its entirety.

For the sake of clarity and understanding, specific physical health tests of a physical exam will now be discussed with respect to blocks 306-310 of the routine 300. A person of ordinary skill in the art will readily recognize that the routine 300 can include one or more other physical health tests in addition to or in lieu of one or more of the physical health tests discussed below. At block 306, the routine 300 continues when a user generates a user's height, weight, temperature, and/or blood data. For example, the software application running on the user's device can instruct the user to manually enter the user's height and/or weight. In these and other embodiments, the software application can instruct the user to use a scale to generate weight and/or body mass index (BMI) data corresponding to the user's weight and/or BMI. In some embodiments, the scale can measure the user's weight and/or BMI and communicate the user's weight and/or BMI to the hub and/or to the software application running on the user's device (e.g., directly or via the hub). In these and still other embodiments, the software application can instruct the user to use a thermometer to generate internal body temperature data corresponding to the user and/or to use a blood testing device (e.g., devices that test for glucose and/or lipids, red blood cell count, white blood cell count, platelets, hemoglobin, hematocrit, calcium, electrolytes, blood enzymes, and/or other characteristics that provide insights on patient health information (e.g., genetic testing, chromosome analysis)) to generate blood data corresponding to the user. In some embodiments, the thermometer can be built into the wearable system. In turn, the thermometer and/or the blood testing device can communicate the user's temperature and/or blood data to the hub and/or to the software application running on the user's device (e.g., directedly or via the hub). In some embodiments, the hub can format the height, weight, BMI, temperature, and/or blood data before sending the formatted data to the software application. For example, the hub can perform one or more calculations (e.g., BMI and/or relative fat mass (RFM)) using the user's height and/or weight data, and can communicate the results of the calculations to the software application. Alternatively, the software application can perform one or more of the calculations once it receives the user's height and/or weight data. In these and other embodiments, the user can review all or a subset of the height, weight, BMI health, temperature, and/or blood data on the software application. In these and other embodiments, the routine 300 can store all or a subset of the height, weight, BMI health, temperature, and/or blood data (e.g., on one or more remote servers and/or databases, and/or associated with the user's account) using the software application and/or the hub.

At block 307, the routine 300 by compressing the wearable system and/or generating a user's body shape and size data. For example, the software application running on the user's device can instruct the user to don the wearable system and/or turn the wearable system on. In some embodiments, a user can press an “on” or “begin” button located on the wearable system (e.g., on the hub) and/or on the software application running on the user's device. In response, the routine 300 can compress the wearable system and/or generate the user's body shape and size data. To compress the wearable system, the routine 300 can use one or more adjustment motors and corresponding adjustment cable(s) in a manner described above with respect to FIGS. 2A and 2B. In these and other embodiments, the routine 300 can track amounts of adjustment cable drawn into the adjustment motor(s) in specific directions to generate a user's body shape and size data. Using this data, the routine 300 can determine a shape and size of a user's body (e.g., a user's torso) and/or can communicate this data to the hub and/or to the software application running on the user's device (e.g., directly or via the hub). In some embodiments, the hub can format the body shape and size data before sending the formatted data to the software application. In these and other embodiments, the user can review all or a subset of the body shape and size data on the software application. In these and still other embodiments, the routine 300 can store all or a subset of the body shape and size data (e.g., on one or more remote servers and/or databases, and/or associated with the user's account) using the software application and/or the hub.

At block 308, the routine 300 continues by generating a user's blood pressure and/or heart rate data. For example, a user can press an “on” or “begin” button located on the wearable system (e.g., on the hub) and/or on the software application running on the user's device. In response, the routine 300 can generate BP/HR data corresponding to the user's blood pressure and/or heart rate using the BP/HR cuffs 221. Alternatively, the routine 300 can automatically proceed to generate the BP/HR data in response to the user's initial and/or prior pressing of the “on” or “begin” button (e.g., at block 307). The routine 300 communicates the user's BP/HR data to the hub and/or to the software application running on the user's device (e.g., directly or via the hub). In some embodiments, the hub can format the BP/HR data before sending the formatted data to the software application. In these and other embodiments, the user can review all or a subset of the BP/HR health data on the software application. In these and other embodiments, the routine 300 can store all or a subset of the BP/HR health data (e.g., on one or more remote servers and/or databases, and/or associated with the user's account) using the software application and/or the hub.

At block 309, the routine 300 continues by generating a user's ECG data. For example, a user can press an “on” or “begin” button located on the wearable system (e.g., on the hub) and/or on the software application running on the user's device. In response, the routine 300 can generate ECG data corresponding to electrical activity of the user's heart using the ECG electrodes 223. Alternatively, the routine 300 can automatically proceed to generate the ECG data in response to the user's initial and/or prior pressing of the “on” or “begin” button (e.g., at blocks 307 and/or 308). The routine 300 communicates the ECG data to the hub and/or to the software application running on the user's device (e.g., directly or via the hub). In some embodiments, the hub can format the ECG data before sending the formatted data to the software application. In these and other embodiments, the routine 300 generates ECG data using each of the ECG electrodes 223 of the wearable system. In these and other embodiments, the routine 300 determines which of the ECG electrodes 223 are positioned at proper locations on a user's body and/or are capturing accurate ECG data. In these embodiments, the routine 300 can generate, communicate, and/or store ECG data collected by only ECG electrodes 223 the routine 300 determines are properly positioned and/or are collecting accurate ECG data. In some embodiments, the user can review all or a subset of the ECG health data on the software application. In these and other embodiments, the routine 300 can store all or a subset of the ECG health data (e.g., on one or more remote servers and/or databases, and/or associated with the user's account) using the software application and/or the hub.

At block 310, the routine 300 continues by generating a user's heart and/or lung activity data. For example, a user can press an “on” or “begin” button located on the wearable system (e.g., on the hub) and/or on the software application running on the user's device. In response, the routine 300 can generate heart and/or lung activity data (e.g., a PCG) using the stethoscope microphones 224. Alternatively, the routine 300 can automatically proceed to generate the heart and/or lung activity data in response to the user's initial and/or prior pressing of the “on” or “begin” button (e.g., at blocks 307, 308, and/or 309). The routine 300 communicates the heart and/or lung activity data to the hub and/or to the software application running on the user's device (e.g., directly or via the hub). In some embodiments, the hub can format the heart and/or lung activity data before sending the formatted data to the software application. In these and other embodiments, the routine 300 generates heart and/or lung activity data using each of the stethoscope microphones 224 of the wearable system. In these and other embodiments, the routine 300 determines which of the stethoscope microphones 224 are positioned at proper locations on a user's body and/or are capturing accurate heart and/or lung activity data. In these embodiments, the routine 300 can generate, communicate, and/or store heart and/or lung activity data collected by only stethoscope microphones 224 the routine 300 determines are properly positioned and/or are collecting accurate heart and/or lung activity data. In some embodiments, the user can review all or a subset of the heart and/or lung activity health data on the software application. In these and other embodiments, the routine 300 can store all or a subset of the heart and/or lung activity health data (e.g., on one or more remote servers and/or databases, and/or associated with the user's account) using the software application and/or the hub.

In some embodiments, all or a subset of the physical health testing devices of or associated with the wearable system can remain powered on and/or connected with the hub for the duration of the physical health exam. Additionally, or alternatively, at the start of one or more of the physical health tests described above with respect to blocks 306-310, the hub can (a) instruct corresponding physical health testing devices to power on and/or (b) attempt to connect with the corresponding physical health testing devices. When the hub successfully connects with the corresponding physical health testing devices, the hub can notify the user device that connection was successful. In some embodiments, the hub can request (and the corresponding physical health testing devices can provide) connection characteristic information (e.g., Bluetooth Low Energy characteristic information). After the hub receives the characteristic information, the hub can subscribe to notifications from the corresponding physical health testing devices (e.g., relating to all or specific user health data generated by the corresponding physical health testing devices). In turn, the corresponding physical health testing devices communicate user health data to the hub. In some embodiments, the hub then disconnects from the corresponding physical health testing devices. The hub can format user health data received from physical health testing devices and/or can communicate the user health data to the user device (e.g., to the software application running on the user device).

At block 311, the routine 300 continues by analyzing the generated health data, generates a physical exam report associated with the user, and/or stores all or a subset of the generated report. For example, the routine 300 (e.g., the individual physical health testing devices, the hub, and/or the software application on the user's device) analyzes all or a subset of the generated health data to identify potential health concerns. In this regard, the routine 300 can compare all or a subset of the generated health data to predetermined “healthy” or “normal” ranges of data. Thus, the routine 300 can indicate in the generated physical exam report which of the physical tests generated health data within a corresponding normal or healthy range of data and which fell outside of a corresponding normal or healthy range of data. In this manner, a user or another individual can review the generated physical exam report and can identify potential health concerns or areas in which further physical health testing is required. In some embodiments, all or a subset of the generated physical exam report can be stored for future reference, analysis, and/or review. For example, the software application running on the user's device and/or the hub can send the generated physical exam report to one or more remote servers to be stored in one or more database entries of one or more databases associated with the user's account.

Additionally, or alternatively, the routine 300 can individually analyze the generated health data received from each physical health testing device of the wearable system (e.g., as the health data is transmitted to the hub and/or to the software application during each physical health test, and/or at the conclusion of the entire physical exam). In these and other embodiments, the routine 300 can generate a plurality of physical test reports that can be displayed to a user (e.g., as the user conducts each physical health test or at the conclusion of the entire physical exam) and/or that can be individually stored for future reference, analysis, and/or review.

In some embodiments, the routine 300 can recommend that a user consult a healthcare professional. For example, the routine 300 can recommend that a user consult a healthcare professional in the event that generated health data falls outside of a corresponding healthy and/or normal range of data. In these and other embodiments, the routine 300 can recommend that a user consult a healthcare professional in the event that a user skips one or more steps of a physical exam and/or in the event of an error when conducting a physical health test of a physical exam. In these and still other embodiments, the routine 300 can recommend specific healthcare professionals or hospitals (e.g., healthcare professionals or hospitals in the user's geographic area, healthcare professionals specializing in a corresponding medical field, etc.).

At block 312, the routine 300 continues by transmitting all or a subset of a user's generated health data to a healthcare professional. In some embodiments, the routine 300 can transmit all or a subset of a user's generated health data report to a healthcare professional at the direction of the user (e.g., via the software application). For example, the routine 300 can transmit all or a subset of the physical exam report generated at block 311. In these and other embodiments, the routine 300 can generate a (e.g., permanent or temporary) code corresponding to the user's account and/or a notification indicating that the user has generated new health data, and the routine 300 can send the code and/or the notification to a healthcare professional. In turn, the healthcare professional can retrieve all or a subset of the health report generated at block 311 using the generated code and/or by responding to the notification (e.g., by logging into an application, website, and/or database associated with the wearable system and entering the code). In these and still other embodiments, the routine 300 can automatically send all or a subset of a user's generated health data to a healthcare professional (e.g., to update the user's medical records) in accordance with the user's prior approval to automatically share the generated health data with the healthcare professional.

At block 313, the routine 300 concludes by returning the physical health testing wearable system to a supplier or vendor. For example, a user can ship the wearable system back to a supplier or vendor (e.g., using a prepaid shipping label included with the wearable system). In these and other embodiments, a user can return the wearable system by returning the system to a location (e.g., a pharmacy or hospital) from which he/she checked-out the wearable system. In some embodiments, the routine 300 can return to block 301 after the routine 300 returns the wearable system to the supplier or vendor.

Although the steps of routine 300 are discussed and illustrated in a particular order, the routine 300 is not so limited. In other embodiments, the routine 300 can perform steps in a different order. In these and other embodiments, any of the steps of the routine 300 can be performed before, during, and/or after any of the other steps of the routine 300. Furthermore, a person of ordinary skill in the art will readily recognize that the routine 300 can be altered and still remain within these and other embodiments of the present technology. For example, steps of the routine 300 in some embodiments can be skipped (e.g., by a user via the software application), such as when an error occurs with a physical health test of the physical exam or for other reasons (e.g., the user experiences needle anxiety when conducting a blood test). Moreover, one or more steps of the routine 300 illustrated in FIG. 3 can be omitted and/or repeated in some embodiments.

Although not shown so as to avoid unnecessarily obscuring the description of embodiments of the technology, any of the forgoing systems and methods described above in FIGS. 1-3 can include and/or be performed by one or more computing devices configured to direct and/or arrange components of the systems and/or to receive, arrange, store, analyze, and/or otherwise process data received, for example, from the machine and/or other components of the systems. As such, such computing devices include the necessary hardware and corresponding computer-executable instructions to perform these tasks. More specifically, computing devices configured in accordance with an embodiment of the present technology can include a processor, a storage device, input/output devices, one or more sensors, and/or any other suitable subsystems and/or components (e.g., displays, speakers, communication modules, etc.). The storage device can include a set of circuits or a network of storage components configured to retain information and provide access to the retained information. For example, the storage device can include volatile and/or non-volatile memory. As a more specific example, the storage device can include random access memory (RAM), magnetic disks or tapes, and/or flash memory.

The computing devices can also include computer readable media (e.g., the storage device, disk drives, and/or other storage media, excluding only a transitory, propagating signal per se) including computer-executable instructions stored thereon that, when executed by the processor and/or computing device, cause the systems to perform physical health testing procedures as described in detail above with reference to FIGS. 1-3. Moreover, the processor can be configured for performing or otherwise controlling steps, calculations, analysis, and any other functions associated with the methods described herein.

In some embodiments, the storage device can store one or more databases used to store data collected by the systems as well as data used to direct and/or adjust components of the systems. In one embodiment, for example, a database is an HTML file designed by the assignee of the present disclosure. In other embodiments, however, data is stored in other types of databases or data files.

One of ordinary skill in the art will understand that various components of the systems (e.g., the computing device) can be further divided into subcomponents, or that various components and functions of the systems may be combined and integrated. In addition, these components can communicate via wired and/or wireless communication, as well as by information contained in the storage media.

### B. ADDITIONAL EXAMPLES

Several aspects of the present technology are set forth in the following examples.
1. A wearable physical health testing system, comprising:
an article of clothing configured to be worn by a user;
a communications hub integrated into the article of clothing; and
a plurality of physical health testing devices integrated into the article of clothing and communicatively coupled to the communications hub,
wherein the plurality of physical health testing devices includes a plurality of electrocardiogram (ECG) electrodes, and
wherein each physical health testing device of the plurality of physical health testing devices is configured to automatically generate physical health data of the user and to transmit generated physical health data to the communications hub.
2. The wearable system of example 1, further comprising an adjustment assembly integrated into the article of clothing and configured to compress the wearable system about the user.
3. The wearable system of example 2, wherein the adjustment assembly includes one or more adjustment motors and one or more adjustment cables.
4. The wearable system of example 3, wherein an adjustment motor of the one or more adjustment motors is configured to track an amount of cable drawn into the adjustment motor in a first direction, and wherein the wearable system is configured to determine a shape and/or a size of a corresponding portion of the user based at least in part on the amount of cable drawn into the adjustment motor in the first direction.
5. The wearable system of any of examples 1-4, wherein the plurality of physical health testing devices includes at least one blood pressure and/or heart rate cuff configured to generate blood pressure and/or heart rate health data of the user.
6. The wearable system of any of examples 1-5, wherein the plurality of ECG electrodes includes one or more redundant ECG electrodes.
7. The wearable system of example 6, wherein the wearable system is configured to determine which ECG electrode of a first group of ECG electrodes of the plurality of ECG electrodes is positioned at a desired location on the user and/or is collecting accurate ECG health data of the user.
8. The wearable system of any of examples 1-7, wherein the article of clothing includes one or more slots or pouches, each configured to receive a gel packet.
9. The wearable system of any of examples 1-8, further comprising a gel packet containing electrically conductive gel, wherein the gel packet is configured to fit within a slot or pouch of the article of clothing, to break when the article of clothing is compressed, and to distribute the electrically conductive gel between an ECG electrode of the plurality of ECG electrodes and the user.
10. The wearable system of any of examples 1-9, wherein the plurality of physical health testing devices includes one or more stethoscope microphones configured to generate heart and/or lung activity health data of the user.
11. The wearable system of example 10, wherein the one or more stethoscope microphones include at least two stethoscope microphones, and wherein the at least two stethoscope microphones include a redundant stethoscope microphone.
12. The wearable system of example 10 or example 11, wherein the one more stethoscope microphones include at least two stethoscope microphones, and wherein the wearable system is configured to determine which stethoscope microphone of a first group of stethoscope microphones of the one or more stethoscope microphones is positioned at a desired location on the user and/or is collecting accurate heart and/or lung activity health data of the user.
13. The wearable system of any of examples 1-12, wherein each physical health testing device of the plurality of physical health testing devices is United States Food and Drug Administration approved.
14. The wearable system of any of examples 1-13, further comprising a user device in wired and/or wireless communication with the communications hub and/or with individual physical health testing devices of the plurality of physical health testing devices, wherein—
the user device includes a display and a software application,
using the display, the software application is configured to present to the user (i) instructions for performing a physical exam using the plurality of physical health testing devices and/or (ii) health data generated by at least one physical health testing device of the plurality of physical health testing devices.
15. The wearable system of any of examples 1-14, further comprising one or more remote servers and/or databases, wherein the communications hub and/or a/the user device is/are configured to transmit all or a subset of the generated physical health data to the one or more remote servers and/or databases for storage in one or more database entries associated with an account of the user.
16. A method for generating a physical exam report of a user, the method comprising:
generating physical health data of the user using a wearable physical health testing system, wherein the wearable system includes an article of clothing, a communications hub integrated into the article of clothing, and a plurality of physical health testing devices integrated into the article of clothing and communicatively coupled to the communications hub wherein—
   wherein the plurality of physical health testing devices includes a plurality of electrocardiogram (ECG) electrodes, and
   each physical health testing device of the plurality of physical health testing devices is configured to automatically generate a corresponding portion of the physical health data of the user and to transmit the corresponding portion of the generated health data to the communications hub;
comparing at least a portion of the generated physical health data of the user to one or more corresponding predetermined ranges of healthy data values; and
based on the comparison(s), generating a physical exam report that includes all or a subset of the generated health data and that indicates any generated physical health data outside of the one or more corresponding predetermined range(s) of healthy data values.
17. The method of example 16, wherein generating the physical health data of the user using the plurality of physical health testing devices includes:
generating blood pressure and/or heart rate health data of the user using a blood pressure and/or heart rate cuff integrated into a sleeve of the article of clothing;
generating ECG health data of the user using the plurality of ECG electrodes; and
generating heart and/or lung activity health data of the user using one or more stethoscope microphones of the plurality of physical health testing devices.
18. The method of example 16 or example 17, further comprising presenting all or a subset of the generated physical health data and/or the generated physical exam report to the user using a software application running on a mobile device of the user.
19. The method of any of examples 16-18, further comprising storing all or a subset of the generated health data and/or the generated physical exam report on one or more remote servers and/or databases in one or more database entries associated with an account of the user.
20. The method of any of examples 16-19, further comprising:
based at least in part on the generated physical exam report, recommending the user consult a healthcare professional;
generating a code associated with the user and all or a subset of the generated physical exam report; and/or
transmitting the code and/or all or a subset of the generated physical exam report to the healthcare professional.

### C. CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order above, alternative embodiments may perform steps in a different order. Furthermore, the various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. To the extent any material incorporated herein by reference conflicts with the present disclosure, the present disclosure controls. Where the context permits, singular or plural terms may also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Furthermore, as used herein, the phrase “and/or” as in “A and/or B” refers to A alone, B alone, and both A and B. Additionally, the terms “comprising,” “including,” “having,” and “with” are used throughout to mean including at least the recited feature(s) such that any greater number of the same features and/or additional types of other features are not precluded.

From the foregoing, it will also be appreciated that various modifications may be made without deviating from the disclosure or the technology. For example, one of ordinary skill in the art will understand that various components of the technology can be further divided into subcomponents, or that various components and functions of the technology may be combined and integrated. In addition, certain aspects of the technology described in the context of particular embodiments may also be combined or eliminated in other embodiments. Furthermore, although advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

What is claimed is:

1. A wearable physical health testing system, comprising:
an article of clothing configured to be worn by a user;
a communications hub integrated into the article of clothing; and
a plurality of physical health testing devices integrated into the article of clothing and communicatively coupled to the communications hub,
wherein the plurality of physical health testing devices includes a plurality of electrocardiogram (ECG) electrodes, and
wherein each physical health testing device of the plurality of physical health testing devices is configured to automatically generate physical health data of the user and to transmit generated physical health data to the communications hub.

2. The wearable system of claim 1, further comprising an adjustment assembly integrated into the article of clothing and configured to compress the wearable system about the user.

3. The wearable system of claim 2, wherein the adjustment assembly includes one or more adjustment motors and one or more adjustment cables.

4. The wearable system of claim 3, wherein an adjustment motor of the one or more adjustment motors is configured to track an amount of cable drawn into the adjustment motor in a first direction, and wherein the wearable system is configured to determine a shape and/or a size of a corresponding portion of the user based at least in part on the amount of cable drawn into the adjustment motor in the first direction.

5. The wearable system of claim 1, wherein the plurality of physical health testing devices includes at least one blood pressure and/or heart rate cuff configured to generate blood pressure and/or heart rate health data of the user.

6. The wearable system of claim 1, wherein the plurality of ECG electrodes includes one or more redundant ECG electrodes.

7. The wearable system of claim 6, wherein the wearable system is configured to determine which ECG electrode of a first group of ECG electrodes of the plurality of ECG electrodes is positioned at a desired location on the user and/or is collecting accurate ECG health data of the user.

8. The wearable system of claim 1, wherein the article of clothing includes one or more slots or pouches, each configured to receive a gel packet.

9. The wearable system of claim 1, further comprising a gel packet containing electrically conductive gel, wherein the gel packet is configured to fit within a slot or pouch of the article of clothing, to break when the article of clothing is compressed, and to distribute the electrically conductive gel between an ECG electrode of the plurality of ECG electrodes and the user.

10. The wearable system of claim 1, wherein the plurality of physical health testing devices includes one or more stethoscope microphones configured to generate heart and/or lung activity health data of the user.

11. The wearable system of claim 10, wherein the one or more stethoscope microphones include at least two stethoscope microphones, and wherein the at least two stethoscope microphones include a redundant stethoscope microphone.

12. The wearable system of claim 10, wherein the one more stethoscope microphones include at least two stethoscope microphones, and wherein the wearable system is configured to determine which stethoscope microphone of a first group of stethoscope microphones of the one or more stethoscope microphones is positioned at a desired location on the user and/or is collecting accurate heart and/or lung activity health data of the user.

13. The wearable system of claim 1, wherein each physical health testing device of the plurality of physical health testing devices is United States Food and Drug Administration approved.

14. The wearable system of claim 1, further comprising a user device in wired and/or wireless communication with the communications hub and/or with individual physical health testing devices of the plurality of physical health testing devices, wherein—
the user device includes a display and a software application,
using the display, the software application is configured to present to the user (i) instructions for performing a physical exam using the plurality of physical health testing devices and/or (ii) health data generated by at least one physical health testing device of the plurality of physical health testing devices.

15. The wearable system of claim 1, further comprising one or more remote servers and/or databases, wherein the communications hub and/or a/the user device is/are configured to transmit all or a subset of the generated physical health data to the one or more remote servers and/or databases for storage in one or more database entries associated with an account of the user.

16. A method for generating a physical exam report of a user, the method comprising:
generating physical health data of the user using a wearable physical health testing system, wherein the wearable system includes an article of clothing, a communications hub integrated into the article of clothing, and a plurality of physical health testing devices integrated into the article of clothing and communicatively coupled to the communications hub wherein—
wherein the plurality of physical health testing devices includes a plurality of electrocardiogram (ECG) electrodes, and
each physical health testing device of the plurality of physical health testing devices is configured to automatically generate a corresponding portion of the physical health data of the user and to transmit the corresponding portion of the generated health data to the communications hub;
comparing at least a portion of the generated physical health data of the user to one or more corresponding predetermined ranges of healthy data values; and
based on the comparison(s), generating a physical exam report that includes all or a subset of the generated health data and that indicates any generated physical health data outside of the one or more corresponding predetermined range(s) of healthy data values.

17. The method of claim 16, wherein generating the physical health data of the user using the plurality of physical health testing devices includes:
generating blood pressure and/or heart rate health data of the user using a blood pressure and/or heart rate cuff integrated into a sleeve of the article of clothing;
generating ECG health data of the user using the plurality of ECG electrodes; and
generating heart and/or lung activity health data of the user using one or more stethoscope microphones of the plurality of physical health testing devices.

18. The method of claim 16, further comprising presenting all or a subset of the generated physical health data and/or the generated physical exam report to the user using a software application running on a mobile device of the user.

19. The method of claim 16, further comprising storing all or a subset of the generated health data and/or the generated physical exam report on one or more remote servers and/or databases in one or more database entries associated with an account of the user.

20. The method of claim 16, further comprising:
based at least in part on the generated physical exam report, recommending the user consult a healthcare professional;
generating a code associated with the user and all or a subset of the generated physical exam report; and/or
transmitting the code and/or all or a subset of the generated physical exam report to the healthcare professional.
